(19) United States
(12) Patent Application Publication
(10) Pub. No.: US 2024/0024696 A1
(43) Pub. Date: Jan. 25, 2024

(54) LIGHT THERAPY WEARABLE

(71) Applicant: Biothread LLC, Wayne, PA (US)

(72) Inventors: Jay Tapper, Wayne, PA (US); Daniel Bish, New York, NY (US); Daniel Shuter, New York, NY (US); Kristien Del Ferraro, Stamford, CT (US)

(21) Appl. No.: 17/867,822

(22) Filed: Jul. 19, 2022

(51) Int. Cl. A61N 5/06; B32B 25/08; B32B 25/20; B32B 25/14; B32B 7/09; B32B 7/12; B32B 3/30; B32B 3/08; B32B 7/023

(52) U.S. Cl. CPC A61N 5/0616; B32B 25/08; B32B 25/20; B32B 25/14; B32B 7/09; B32B 7/12; B32B 3/30; B32B 3/08; B32B 7/023; B32B 2250/03; B32B 2250/24; B32B 2307/416; B32B 2307/4026; B32B 2307/536; B32B 2307/732; B32B 2535/00; A61N 2005/0645; A61N 2005/0659; A61N 2005/0663

(57) ABSTRACT

A light therapy wearable includes a fabric panel, a light-transmissive pad and a side-emitting optical fiber. The fabric panel includes an inner surface that when the wearable is worn is configured to face a wearer's skin. The light-transmissive pad connects with the fabric panel and at least partially covering the inner surface. The light-transmissive pad can be made from a light-transmissive material having a durometer of at least 50 A. At least a portion of the side-emitting optical fiber can sandwiched between the inner surface of the fabric panel and the light-transmissive pad.

### BACKGROUND

Clinical studies have demonstrated the ergogenic and prophylactic benefits of red and infrared light therapy. Red and infrared light have been found to increase blood flow to muscles and joints, which can create an anti-inflammatory response, in addition to providing increased pliability. Muscle and joint stiffness as well as soreness have been demonstrated to be significantly reduced while muscle contractile function is simultaneously improved by using red and infrared light.

Clothing made from light emitting fabrics is described in U.S. Pat. No. 4,234,907. This patent, however, describes such clothing as a fad item or as safety clothing to emit light outward when the wearer wishes to be seen by others. US 2013/0116612 A1 discloses an illuminatable pad in FIG. 4 and different types of braces or supports in FIGS. 10-13. The pads, braces and supports disclosed in US 2013/0116612 A1 are described as including elongated polymeric tubes sold by Poly Optics Australia, Pty Ltd, which according to their literature have a gel core made from optically pure case acrylic monomers with a smallest available outer diameter (OD) for the core being 3.0 mm. Poly Optics Australia, Pty Ltd also sells the core placed in a jacket, which is described as a “Poly Jacket,” and the smallest available OD when provided in the jacket according to the available literature is 5.0 mm.

There are problems that need to be overcome to provide a light therapy wearable that incorporates side-emitting optical fibers. The literature available from manufacturers of side-emitting optical fibers warns against bending the fiber in a tight arc, and further warns against excessive force, repetitive bending and dropping. Also, when relatively larger optical fibers are used in wearables, as is the case in US 2013/0116612 A1, spacing between adjacent sections of the light tube must be increased to accommodate for the minimum bend radius of the optical fiber. There are also wearer comfort issues that result from the use of relatively larger optical fibers. US 2013/0116612 A1 attempts to overcome these comfort issues by changing the cross section of the optical fiber from a circular cross section to one that is more elliptical with tapered side edges (see FIGS. 14 and 14A in US 2013/0116612 A1). Also, US 2013/0116612 A1 describes placing the light tube in a foam to prevent discomfort for the user. These comfort and wearability problems, among others, need overcome to provide a consumer a light therapy wearable that can be handled the same as or nearly the same as a typical wearable that does not include side-emitting optical fibers.

### SUMMARY

In view of the foregoing, a light therapy wearable includes a fabric panel, a light-transmissive pad and a side-emitting optical fiber. The fabric panel includes an inner surface that when the wearable is worn is configured to face a wearer's skin. The light-transmissive pad connects with the fabric panel and at least partially covers the inner surface. The light-transmissive pad can be made from a light-transmissive elastomer having a durometer of at least 50 A. At least a portion of the side-emitting optical fiber can sandwiched between the inner surface of the fabric panel and the light-transmissive pad.

A method of manufacturing a light therapy wearable includes placing a side-emitting optical fiber on an inner surface of a fabric panel. The inner surface is configured to face a wearer's skin when the wearable is worn. The method further includes at least partially covering the side-emitting optical fiber with a light-transmissive pad being made from a light-transmissive elastomer and having a durometer of at least 50 A.

### BRIEF DESCRIPTION OF THE DRAWINGS

FIG. 1 is a perspective view of a light therapy wearable.

FIG. 2 is an exploded view of the light therapy wearable depicted in FIG. 1.

FIG. 3 is an upper perspective view of an alternative light-transmissive pad for the light therapy wearable depicted in FIG. 1.

FIG. 4 is a lower perspective view of the alternative light-transmissive pad depicted in FIG. 3.

FIG. 5 is a cross-sectional view showing a portion of the light therapy wearable depicted in FIG. 1 folded over itself.

FIG. 6 is a cross-sectional view of a portion of the light-transmissive pad depicted in FIGS. 3 and 4.

### DETAILED DESCRIPTION

Referring to FIG. 1, a light therapy wearable 20 includes a fabric panel 22, a light-transmissive pad 24 and a side-emitting optical fiber 26. The light therapy wearable 20 is useful to provide light therapy to one who wears the light therapy wearable 20. The light therapy wearable 20 is configured to project light, which can be of a therapeutic wavelength, toward a person or animal wearing the light therapy wearable 20. The light therapy wearable 20 can be configured to project light toward targeted body areas, which can include particular muscles, muscle groups, joints, human extremities, and the wearer's skin as examples. The light therapy wearable 20 shown in FIG. 1 is shown as a pad to be worn by a person by being placed on, around or over a wrist, arm, ankle, leg, knee, etc. Fasteners (not shown) such as buckles or hook and loop fasteners can be provided on the light therapy wearable 20 to facilitate wearing the light therapy wearable 20. Additional to or in lieu of fasteners, the light therapy wearable 20 can be part of an elastic element, e.g., part of an elastic brace, that is pulled on by the wearer similar to known knee, elbow and ankle braces.

The fabric panel 22 can be made from a neoprene micro-cell structure similar to known compression sleeves. The fabric panel 22 can also be made from a fabric panel similar to a cut and sew fabric pattern piece or fully fashioned knitted structure. Where the fabric panel 22 is made from a cut and sew fabric pattern piece or fully fashioned knitted structure, the fabric panel 22 can be affixed, e.g., sewn to a stretchable component, e.g., a neoprene micro-cell structure similar to known compression sleeves and braces. The fabric panel 22 should be made so that it generally conforms to the wearer when the light therapy wearable 20 is being worn.

The fabric panel 22 includes an inner surface 32 that when the light therapy wearable 20 is worn is configured to face a wearer's skin. The fabric panel 22 also includes an outer surface 34 that is opposite to the inner surface 32. The inner surface 32 can be reflective of red light and infrared light. For example, the fabric panel 22 can be a red dyed or white fabric panel that will reflect light impinging on the inner surface 32 back toward the wearer. A peripheral edge 36 of the fabric panel 22 is shown as square shaped in FIG. 1; however, the peripheral edge 36 can take any number of different shapes.

The light-transmissive pad 24 connects with the fabric panel 22 and at least partially covers the inner surface 32. The light-transmissive pad 24 is made from a light-transmissive material. In the illustrated embodiment the light-transmissive pad 24 is made from a light-transmissive elastomer having a durometer of at least 50 A, such as solid silicone having a durometer of at least 50 A. By making the light-transmissive pad 24 from a light-transmissive elastomer having a durometer of at least 50 A the light-transmissive pad 24 can feel comfortable when the first surface 42 of the light-transmissive pad 24 contacts the wearer's skin. By making the light-transmissive pad 24 from a light-transmissive solid silicone, in contrast, to a closed cell (e.g., sponge) or open cell (e.g., foam) structure, more protection is provided to the side-emitting optical fiber 26 as compared a closed cell or open cell pad. The light-transmissive pad 24 can be made from other light-transmissive polymers, e.g., polyurethane. The light-transmissive pad 24 includes a first surface 42 that when the light therapy wearable 20 is worn is configured to face a wearer's skin and a second surface 44 that faces away from the wearer's skin and toward the inner surface 32 of the fabric panel 22 when the light therapy wearable 20 is worn. The light-transmissive pad 24 also includes an outer peripheral edge 46, which can be offset from the peripheral edge 36 of the fabric panel 22 when the light therapy wearable 20 is assembled. The light-transmissive pad 24 also includes an inner edge 52 that defines an opening 54. The light-transmissive pad 24 can be provided with more than one opening extending through the light-transmissive pad 24 between the first surface 42 and the second surface 44, and those openings could be provided in locations of the light-transmissive pad 24 that would normally cover the side-emitting optical fiber 26 such that only portions of the side-emitting optical fiber 26 are covered by the light-transmissive pad 24. It can be desirable, however, to size and number these openings so that the side-emitting optical fiber 26 is still offset from the wearer's skin when the light therapy wearable 20 is worn.

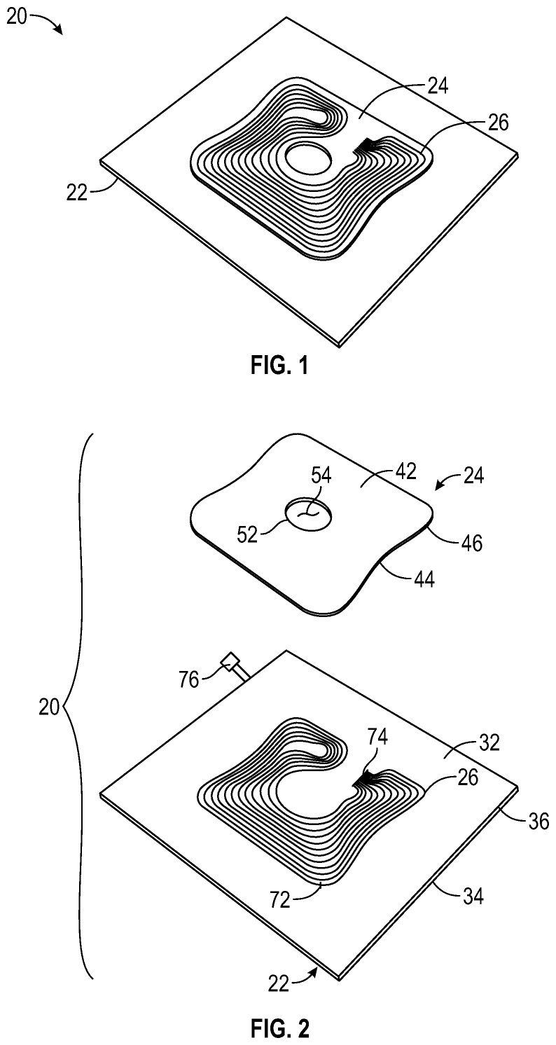

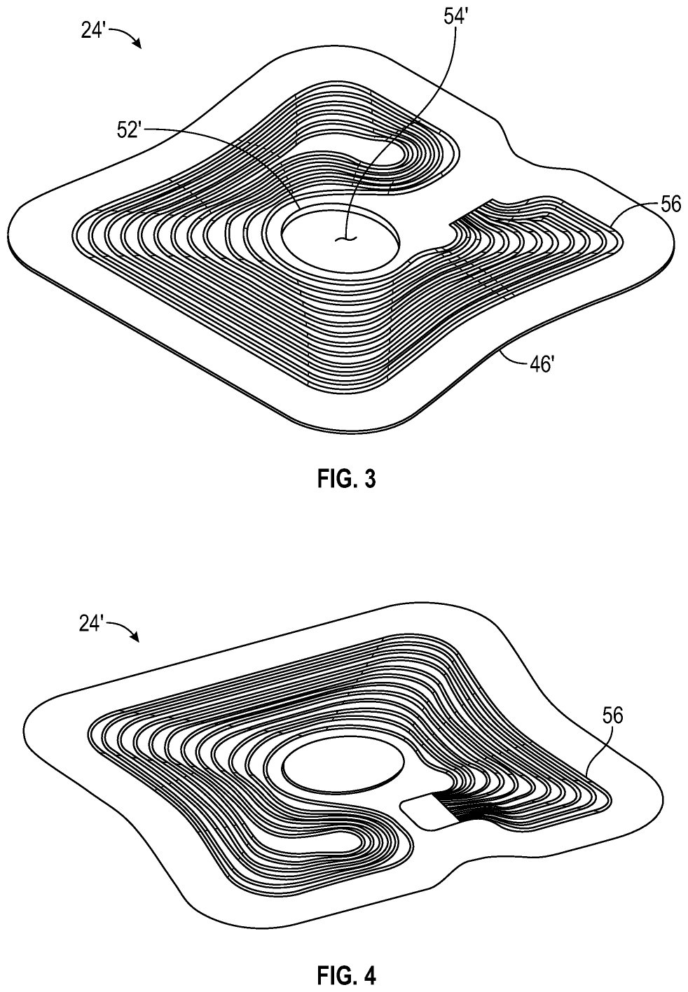

The light-transmissive pad 24 depicted in FIG. 2 is depicted as including a smooth first surface 42 and a smooth second surface 44. The light-transmissive pad 24 depicted in FIG. 2 has a constant thickness measured between the first surface 42 and the second surface 44 throughout the light-transmissive pad 24. FIGS. 3 and 4 depict an alternative light-transmissive pad 24′. For ease of understanding this alternative, like components are designated by like numerals with a primed (′) suffix and new components are designated by new numerals. The light-transmissive pad 24′ can be molded to include to include a channel 56 or channels that match the pattern of the side-emitting optical fiber 26 on the inner surface 32 of the fabric panel 22. For example, the light-transmissive pad 24′ in the FIGS. 3 and 4 is formed having at least one channel 56 that receives a portion of the side-emitting optical fiber 26 sandwiched between the inner surface 32 of the fabric panel 22 and the light-transmissive pad 24. The light-transmissive pad 24′ depicted in FIGS. 3, 4 and 6 may not have a constant thickness measured between the first surface 42′ and the second surface 44′ throughout the light-transmissive pad 24′. With either the light-transmissive pad 24 or the light-transmissive pad 24′, at least a portion of the side-emitting optical fiber 26 is sandwiched between the inner surface 32 of the fabric panel 22 and the light- transmissive pad 24, 24′.

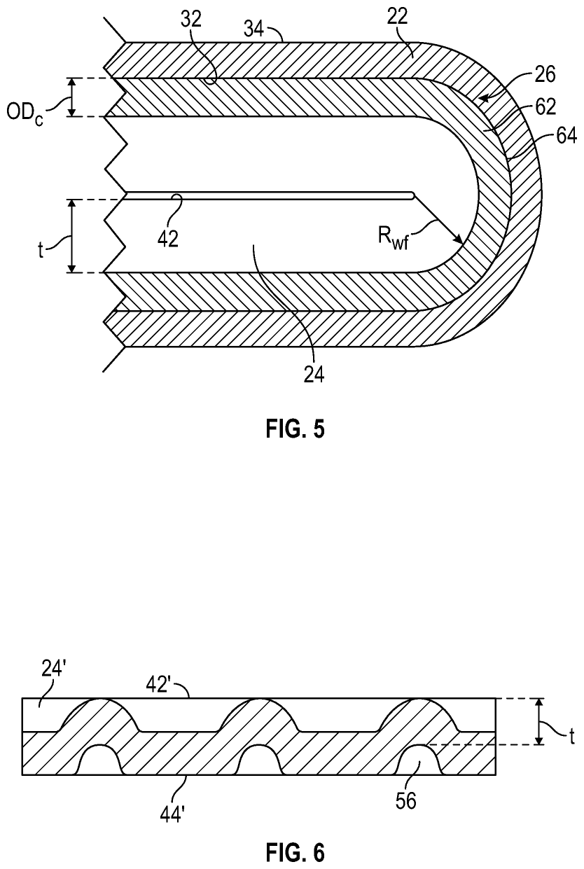

With reference to FIG. 5, the side-emitting optical fiber 26 includes a core 62 and cladding 64 surrounding the core 62. The core 62 in the illustrated embodiment is made from a synthetic polymeric material, e.g., a high-purity polymethyl methacrylate (PMMA). The cladding 64 in the illustrated embodiment is made from a fluorinated polymer. As seen in FIG. 5, at least a portion of the second surface 44 of the light-transmissive pad 24 and the inner surface 32 of the fabric panel 22 are in contact with the side-emitting optical fiber 26. As mentioned above, there are difficulties with incorporating side-emitting optical fiber into a wearable in an aesthetic and practically functional manner. If the side-emitting optical fiber 26 cannot withstand bending and forces that would be applied to the side-emitting optical fiber 26 during typical wear and handling of the light therapy wearable 20, then this would limit the practical functionality in that the light therapy wearable 20 would need to be handled much differently than a typical wearable not having any side-emitting optical fiber.

FIG. 5 depicts the light therapy wearable 20 folded over itself. In FIG. 5, the side-emitting optical fiber 26 has a core diameter (OD_{C}) of 0.5 mm. A thickness (t) of the light-transmissive pad 24 between the first surface 42 and a location of the side-emitting optical fiber 26 nearest to the first surface 42 is at least two times the core diameter of the side-emitting optical fiber 26. For example, in the embodiment depicted in FIG. 5 the thickness (t) of the light-transmissive pad 24 between the first surface 42 and the location of the side-emitting optical fiber 26 nearest to the first surface 42 is at least 1.05 mm. Available literature from optical fiber manufacturers recommends that the radius of the arc in which a side-emitting optical fiber is bent should not be less than 20 times the core diameter (OD_{C}) of the side-emitting optical fiber; however, this radius is likely based on maintaining a desired performance for the side-emitting optical fiber in its capacity to transmit light. There is another minimum radius of an arc in which a side-emitting optical fiber is bent that will result in permanent mechanical damage, e.g., the side-emitting optical fiber 26 can break or crack so that light can no longer travel through the side-emitting optical fiber 26 at the location of the permanent mechanical damage. With reference to FIGS. 5 and 6, the thickness (t) of the light-transmissive pad 24 between the first surface 42 and a location of the side-emitting optical fiber 26 nearest to the first surface 42 is sized with respect to the core diameter (OD_{C}) of the side-emitting optical fiber 26 such that the minimum bend radius when folded (R_{WF}) for the side-emitting optical fiber 26 is greater than the minimum radius of the arc in which the side-emitting optical fiber 26 is bent that will result in permanent mechanical damage to the side-emitting optical fiber 26. The thickness (t) of the light-transmissive pad 24 being at least two times the core diameter of the side-emitting optical fiber 26 provides a large enough bend radius for the side-emitting optical fiber 26 to inhibit permanent mechanical damage. Also, the thickness (t) of the light-transmissive pad 24 can be less than five times the core diameter of the side-emitting optical fiber 26 to minimize the thickness of the light-transmissive pad 24, which can provide for a relatively light weight light therapy wearable 20. That the light-transmissive pad 24 can be made from a light-transmissive solid elastomer can also further protect the side-emitting optical fiber 26 as the light therapy wearable 20 is being handled in a fashion similar to known wraps and garments.

The light-transmissive pad 24, 24′ can be formed, e.g., molded into the configuration shown in FIG. 2, FIGS. 3 and 4 or another shape and then be affixed to the fabric panel 22. For example, light-transmissive pad 24, 24′ can be sewn to the fabric panel 22 adjacent to the outer peripheral edge 46 and adjacent to the inner edge 52, 52′, which defines an opening 54, 54′ in the light-transmissive pad 24, 24′. Alternatively, the light-transmissive pad 24, 24′ can be affixed to the fabric panel 22 with an adhesive. When the light-transmissive pad 24, 24′ is made from silicone, the light-transmissive pad 24, 24′ can be poured in a semi-liquid state onto the fabric panel 22. Silicone does not chemically bond well to many materials but it forms mechanical bonds between the fibers of the fabric panel 22 when it solidifies.

The side-emitting optical fiber 26 can be fixed to the inner surface 32 of the fabric panel 22. For example, the side-emitting optical fiber can be sewn or embroidered to the inner surface 32. FIG. 2 only depicts one such stitch 72 for sewing or embroidering the side-emitting optical fiber to the inner surface 32 for clarity purposes, but many more similar stitches could be used to fix side-emitting optical fiber 26 the side-emitting optical fiber 26 to the inner surface 32 of the fabric panel 22. The side-emitting optical fiber 26 can also be placed on the inner surface 32 of the fabric panel 22 and fixed to the inner surface 32 via the light-transmissive pad 24, 24′, for example when the light-transmissive pad 24, 24′ is poured onto the fabric panel 22. A slit 74 or similar opening can be provided in the fabric panel 22 through which the side-emitting optical fiber 26 passes to allow the side-emitting optical fiber 26 to pass from the inner surface 32 toward the outer surface 34. When a plurality of for side-emitting optical fibers 26 are provided on the inner surface 32 of the fabric panel 22 they can be bundled into a connector 76 (depicted schematically in FIG. 2) for connection to a light source (not shown) that can attach to the outer surface of the light therapy wearable 20, which may or may not be the outer surface 34 of the fabric panel 22.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A light therapy wearable comprising:
a fabric panel including an inner surface that when the wearable is worn is configured to face a wearer's skin;
a light-transmissive pad connected with the fabric panel and at least partially covering the inner surface, the light-transmissive pad being made from a light-transmissive elastomer and having a durometer of at least 50 A; and
a side-emitting optical fiber, at least a portion of the side-emitting optical fiber being sandwiched between the inner surface of the fabric panel and the light-transmissive pad.

2. The light therapy wearable of claim 1, wherein the side-emitting optical fiber is fixed to the inner surface via the light-transmissive pad.

3. The light therapy wearable of claim 1, wherein the side-emitting optical fiber is sewn or embroidered to the inner surface.

4. The light therapy wearable of claim 1, wherein the light-transmissive pad is formed having at least one channel that receives the portion of the side-emitting optical fiber sandwiched between the inner surface of the fabric panel and the light-transmissive pad.

5. The light therapy wearable of claim 4, wherein the light-transmissive pad includes a first surface that when the wearable is worn is configured to face a wearer's skin, wherein a thickness of the light-transmissive pad between the first surface and a location of the side-emitting optical fiber nearest to the first surface is at least two times a core diameter of the side-emitting optical fiber.

6. The light therapy wearable of claim 5, wherein the thickness of the light-transmissive pad between the first surface and the location of the side-emitting optical fiber nearest to the first surface is less than five times the core diameter of the side-emitting optical fiber.

7. The light therapy wearable of claim 1, wherein the light-transmissive pad includes a first surface that when the wearable is worn is configured to face a wearer's skin, wherein first surface is smooth.

8. The light therapy wearable of claim 7, wherein the light-transmissive pad includes a second surface opposite the first surface, wherein the light-transmissive pad has a constant thickness measured between the first surface and the second surface throughout the light-transmissive pad.

9. The light therapy wearable of claim 1, wherein the light-transmissive pad includes a first surface that when the wearable is worn is configured to face a wearer's skin, wherein a thickness of the light-transmissive pad between the first surface and a location of the side-emitting optical fiber nearest to the first surface is at least two times a core diameter of the side-emitting optical fiber.

10. The light therapy wearable of claim 9, wherein the light-transmissive pad is a solid elastomer.

11. The light therapy wearable of claim 1, wherein the light-transmissive pad is a solid elastomer.

12. The light therapy wearable of claim 1, wherein the light-transmissive pad includes a first surface that when the wearable is worn is configured to face a wearer's skin and a second surface opposite the first surface, wherein at least a portion of the second surface of the light-transmissive pad and the inner surface of the fabric panel are in contact with the side-emitting optical fiber.

13. The light therapy wearable of claim 1, wherein the light-transmissive pad is sewn to the fabric panel.

14. The light therapy wearable of claim 1, wherein the light-transmissive pad is affixed to the fabric panel with adhesive.

15. The light therapy wearable of claim 1, wherein the light-transmissive pad is silicone and affixed to the fabric panel via mechanical bonds between fibers of the fabric panel and the silicone.

16. The light therapy wearable of claim 1, wherein the inner surface of the fabric panel is reflective of red light and infrared light.

17. The light therapy wearable of claim 1, wherein the fabric panel is a red dyed fabric panel.

18. The light therapy wearable of claim 1, wherein the fabric panel is a white fabric panel.

19. A method of manufacturing a light therapy wearable, the method comprising:
placing a side-emitting optical fiber on an inner surface of a fabric panel, the inner surface being configured to face a wearer's skin when the wearable is worn; and
at least partially covering the side-emitting optical fiber with a light-transmissive pad being made from a light-transmissive elastomer and having a durometer of at least 50 A.

20. The method of claim 19, further comprising sewing or embroidering the side-emitting optical fiber to the inner surface prior to at least partially covering the side-emitting optical fiber with the light-transmissive pad.

21. The method of claim 19, wherein at least partially covering the side-emitting optical fiber with the light-transmissive pad includes pouring a silicone onto the inner surface of the fabric panel.

22. The method of claim 19, wherein at least partially covering the side-emitting optical fiber with the light-transmissive pad includes contacting at least a portion of the light-transmissive pad with the side-emitting optical fiber.
